(19) United States
(12) Patent Application Publication
(10) Pub. No.: US 2007/0054886 A1
(43) Pub. Date: Mar. 8, 2007

(54) Ras antagonists for treating neurodegenerative disorders

(75) Inventors: Yoel Kloog, HERZLIA (IL); Esther Shohami, Mevasseret Zion (IL)

Correspondence Address: LERNER, DAVID, LITTENBERG,;KRUMHOLZ & MENTLIK, 600 SOUTH AVENUE WEST, WESTFIELD, NJ, 07090, US

(73) Assignee: RAMOT AT TEL AVIV UNIVERSITY, LTD., TEL-AVIV (IL)

(21) Appl. No.: 10/558,132

(22) PCT Filed: May 21, 2004
(86) PCT No.: PCT/IB04/02294

§ 371 Date: Nov. 23, 2005

Related U.S. Application Data
(60) Provisional application No. 60/472,835, filed on May 23, 2003.
(60) Provisional application No. 60/475,667, filed on Jun. 4, 2003.

(51) Int. Cl. A61K 31/655; A61K 31/60; A61K 31/195; A61K 31/192

(52) U.S. Cl. 514/151; 514/345; 514/159; 514/568; 514/567

(57) ABSTRACT

Disclosed are methods of neuroprotection or treatment of neurodegenerative disorders with Ras antagonists.

### CROSS-REFERENCE TO RELATED APPLICATIONS

This application is a national stage entry of International Application No. PCT/IB2004/002294, filed May 21, 2004, which claims the benefit of priority under 35 U.S.C. § 119(e) on the basis of U.S. Provisional applications 60/472,835, filed May 23, 2003, and 60/475,667, filed Jun. 4, 2003, the contents of which are hereby incorporated by reference in their entireties.

### BACKGROUND OF THE INVENTION

Pathological conditions resulting from death of nerve cells (also referred to as neurons) in the central -nervous system are prevalent in our society and include acute and chronic neurodegenerative disorders. Examples of such disorders include Alzheimer's disease, stroke, ischemia, anoxia, hypoxia, Wernicke-Kosakoff's related dementia (alcohol induced dementia), hematoma, traumatic brain or head injury, and epilepsy. Other examples of neurodegenerative disorders include Parkinson's disease, Huntington's disease, AIDS Dementia, age related dementia, age-associated memory impairment, hypoglycemia, cerebral edema, arteriosclerosis, spinal cord cell loss, and peripheral neuropathy. Chronic and acute neurodegeneration have been largely untreatable with previous methods. Patients' disability resulting from these conditions can cause a significant reduction in quality of life.

Major causes of neuronal cell death following brain insults are the large excess of glutamate, the major excitatory amino acid in the brain, and its action on glutamate receptors. Among these receptors, the N-methyl-D-aspartate (NMDA) receptors (NMDAR) play a major role in glutamate toxicity (also named excitotoxcity). Activation of NDMAR by glutamate leads to a large entry of Ca²⁺ ions into the neurons resulting in their death. It is believed that NMDAR-mediated toxicity stems from the large excesses of Ca²⁺ ions in the nerve cells, and the involvement of critical components that are activated by these ions, including protein kinase C (PKC) isoforms and mitogen activated protein kinase (MAPK) cascades.

One of the first events associated with excitotoxicity and nerve cell death is activation of PKC. PKC refers to a family of more than 10 Ca²⁺/phospholipid-dependent and independent threonine-serine kinase isozymes which regulate a multitude of mechanisms including cell differentiation and response to injury. PKCs are abundant in neurons. It has been established that ischemia affects PKC activity and distribution. Ischemic nerve cell death has been associated with induction of PKC-delta isozyme, an effect that can be blocked by NMDA inhibitors. Increased PKC-gamma immunoreactivity following incomplete ischemia has been found in the hippocampus. It has been shown that NMDAR stimulation can trigger PKC-gamma and beta isozyme activation.

Several PKC isozymes (for example, PKC-delta and epsilon) activate the mitogen-activated protein kinase (MAPK) cascade. The MAPK family consists of key regulatory proteins that are known to regulate cellular responses to both proliferative and stress signals. MAPK is abundantly expressed in nerve cells and may be necessary for cellular commitment to apoptosis. Apoptosis, also known as “programmed cell death”, is a mechanism of nerve cell death initiated by activation of intracellular enzymes known as caspases. When a cell undergoes apoptosis, its membrane disintegrates, exposing the inside of the membrane's lipid bilayer. MAPKs consist of several enzymes, including a subfamily of extracellular signal-activated kinases (ERK1 and ERK2) and stress-activated MAPKs. There are three distinct groups of MAPKs in mammalian cells, namely: a) extracellular signal-regulated kinases (ERKs); b) c-Jun N-terminal kinases (JNKs); and c) stress activated protein kinases (SAPKs). An illustration of the MAPK cascade can be described as follows. PKC activation or other factors (e.g. increases in free intracellular Ca²⁺) activates small proteins called Ras and Raf-1, which in turn activate MAPK/ERK kinases referred to as MEKs. The MEKS in turn activate ERKs. The ERKS translocate to the cell nucleus where they activate transcription factors and thereby regulate cell proliferation. The inhibition of these protein kinases produces neuroprotective and neuron-treating effects as does the inhibition of the MAPK cascade. Examples of such kinases are mitogen-activated protein kinase 1 and 2, their homologues and isoforms, extracellular signal-regulated kinases (ERKs) their homologues and isoforms (ERK1, ERK2, ERK3, ERK4), and a group of kinases known as MAP/ERK kinases 1 and 2 or MEK1/2. Exposure of cells to stress activates protein kinases by a variety of mechanisms. For example, ischemia, NMDA and amyloid peptides all activate MAPK. Studies of functional roles of MAPKs in nerve tissue suggest that MAPK could be an important regulator of nerve cell death and plasticity.

In its active (GTP-bound) state, Ras activates a multitude of effector molecules associated with regulation of cell growth and differentiation, cell death and survival, and cell adhesion and migration (Shields et al., 2000). Ras-GTP is formed by receptor-mediated activation of guanine nucleotide exchange factors (GEFs) that induce an exchange of GDP for GTP, whereupon the signal is turned off by GTPase-activating proteins (Scheffzek et al., 1997). The classic Ras pathway is the one in which growth factors induce activation of Ras that activates the Raf/MEK/extracellular signal-regulated kinase (ERK) mitogen activated protein kinase (MAPK) cascade (Shields et al., 2000).

Calcium influx through the NMDAR also activates the Ras/ERK pathway (Chen et al., 1998). ERK is associated with NMDAR functions (Atkins et al., 1998; Brambilla et al., 1997; English and Sweatt, 1996; Fukunaga and Miyamoto, 1998; Kaminska et al., 1999; Rosenblum et al., 1997). Active Ras and MAPKs also participate in neuroinflammatory responses (Dalakas, 1995) and in excitotoxicity (Ferrer et al., 2002).

Traumatic brain injury is a leading cause of mortality and disability in individuals and accounts for an estimated 2 million new cases per year in the USA (Sosin et al., 1995). The primary mechanical impact activates cellular and molecular responses that lead to active processes mediated by many biochemical pathways, including Ras pathways, resulting in secondary damage (for review see Kochanek et al., 2000). Studies of traumatic brain injury in experimental animal models have contributed to the understanding of the pathophysiology of this condition (Laurer and McIntosh, 1999). Excessive glutamate release, which occurs within minutes of trauma, activates various subtypes of glutamate receptors and contributes to injury processes (Faden et al., 1989). A post-traumatic decrease in binding of ligands to NMDAR has also been reported (Miller et al., 1990; Sihver et al., 2001). Neuroinflammation (including cytokine production, reactive astrocytosis, microglial activation, and macrophage infiltration) is another of the early responses to traumatic brain injury (Feuerstein et al., 1998; Shohami et al., 1999; Morganti-Kossmann et al. 2000). It has been shown that neuroinflammation, like excessive glutamate release, is also associated with a significant decrease in NMDAR (Biegon et al., 2002).

### SUMMARY OF THE INVENTION

Aspects of the present invention are directed to treatment of acute or chronic disease, trauma or aging, collectively referred to herein as “neurodegenerative disorders,” by administering to an animal (e.g., mammal such as a human) in need thereof, an effective amount of a Ras antagonist. Thus, one aspect of the present invention is directed to a method of neuroprotection, reduction of a neurological deficit or protection of nerve cells from deterioration and cell death arising from a neurodegenerative disorder, by administering to an animal in need thereof, an effective amount of a Ras antagonist. A related aspect of the present invention is directed to a method for reducing levels of Ras-GTP or reducing loss of NMDAR binding associated with a neurodegenerative disorder, by administering to an animal in need thereof, an effective amount of a Ras antagonist. In some preferred embodiments, the neurodegenerative disorder is mediated by glutamate toxicity. Such embodiments include treatment of acute head or brain trauma or injury, ischemia and stroke. Other preferred embodiments of the present invention entail the administration of Ras antagonists that include farnesyl-thiosalicylic acid (FTS) (e.g., S-trans, trans-FTS) and its analogs.

Ras antagonists useful in the present invention have been reported as being useful in the treatment of cancer and non-malignant diseases characterized by ras-mediated proliferation of cells, such as autoimmune diseases. The present invention is based on the discovery that ras antagonists provide a therapeutic effect in connection with neurodegenerative disorders (e.g., neurodegenerative disorders involving glutamate-mediated toxicity such as traumatic head or brain injury, ischemia and stroke) that involve non-dividing, differentiated nerve cells.

### BRIEF DESCRIPTION OF THE DRAWINGS

FIG. 1 is a graph showing the time course of [³H]-FTS accumulation in the mouse brain. Mice received [³H]-FTS (3 mg/kg, i.p.) and the amounts of labeled drug in the forebrain were then determined at the indicated times. Mean values (dpm/g tissue) of two separate determinations at each time point are shown.

FIG. 2 is an immunoblot showing the closed head injury (CHI) induced increase in Ras-GTP and in phospho-ERK in the contused hemisphere. The brains of either sham mice (n=3) or of CHI mice at the indicated times after the injury (n=3) were removed and the left (contused) hemispheres were used for immunoblotting assays. The amounts of total Ras and Ras-GTP were then determined by immunoblotting with pan-Ras antibody and the amounts of total ERK and phospho-ERK were determined by immunoblotting with anti-ERK and anti phospho-ERK Ab.

FIG. 3 is a chart and accompanying immunoblot showing a transient increase in Ras-GTP induced in the brain by CHI and inhibited by treatment with the Ras inhibitor FTS. One hour after CHI, mice were treated with the vehicle (CHI) or with 5 mg/kg FTS (CHI+FTS). The mice were killed 2 hrs or 24 hrs after the injury (n=4 per group). The amounts of total Ras and Ras-GTP were then determined in both the left (contused, L) and the right (R) hemispheres by immunoblotting with pan-Ras antibody. Sham-injured mice received vehicle but were not injured, and their representative immunoblots are shown (a). The increase in Ras-GTP 2 hrs after CHI was 3.8±0.3 fold higher (mean±SD) in the left (contused) hemisphere (P=0.01) and 1.6±0.2 fold higher in the right hemisphere (P=0.05) than in sham-injured mice (b). The estimated degree of inhibition of the increase in Ras-GTP by FTS at this time point (mean±SD, CHI vs. CHI+FTS) was 81.7±10.0% (left hemisphere; P<0.01) and 70.4±12.0% (right hemisphere; P<0.05) as shown in panel c.

FIG. 4 is a chart and accompanying immunoblot showing MK-801, like FTS, reduces the amounts of Ras-GTP and phospho-ERK in the brains of CHI mice. One hour after CHI, mice were treated with the vehicle (CHI) or with 0.5 mg/kg MK-801 (CHI+MK-801) or with 5 mg/kg FTS (CHI+FTS). The mice were killed 2 hrs after the injury (n=4 per group). The amounts of total Ras and Ras-GTP were then determined in both left (contused, L) and right (R) hemispheres, by immunoblotting with pan-Ras antibody (a, upper panel). The extent of inhibition by MK-801 at this time point (CHI vs. CHI+MK-801) was 53.5±8.3% (mean±SD) in the left (contused) hemisphere (P=0.004) and 31.2±4.9% in the right hemisphere (P=0.02) as shown (a, lower panel). The corresponding values for inhibition by FTS (CHI vs. CHI+FTS) were 75.2±6.8% (left, P=0.001) and 67.2±7.1% (right, P=0.0004). The inhibition induced by FTS was significantly stronger than that induced by MK-801 (P=0.04 and 0.003, respectively, for left and right hemispheres). (b) The amounts of total ERK and phospho-ERK were determined by immunoblotting with anti-ERK and anti phospho-ERK Ab. The experiment was performed in quadruplicate, and representative immunoblots are shown. The extent of inhibition by MK-801 (CHI vs. CHI+MK-801) was 45.8±8.4% in the left (contused) hemisphere (P=0.003) and 42.7±4.8% in the right hemisphere (P=0.0002) as shown in the lower panel of b. The corresponding values for inhibition by FTS (n=4) were 72.8±9.8% (P=0.006) and 73.2±8.3% (P=0.002). The inhibition induced by FTS was significantly stronger than that induced by MK-801 (P=0.0002 and 0.011, respectively, for left and right hemispheres).

FIG. 5 is a pseudo-colored autoradiographic image showing the prevention of long-term loss of NMDAR binding by FTS. Mice were injected with FTS or vehicle 1 hour after CHI, evaluated for neurological deficits for 7 days, and then decapitated. Their brains were sectioned for quantitative autoradiography and histology and pseudo-colored (rainbow spectrum, purple=low, red=high) images are shown. The top image is from a sham animal, showing symmetrical MK-801 binding. The middle image is from traumatized mouse at the same anatomical level, just posterior to the lesion, showing profound loss of NMDAR binding relative to the contralateral hemisphere in the cortex and striatum. The bottom image shows a section at the same anatomical level from an FTS-treated mouse, with symmetrical binding of MK-801 indicating preservation of the NMDAR.

FIG. 6 is a magnified image showing the effect of FTS on lesion size after CHI. Brain sections used for autoradiography on day 7 post-CHI (FIG. 5) were stained with cresyl violet and magnified at low power (4×). The illustration shows a section through the area of maximal lesion in one vehicle treated CHI mouse (A) and a section through the same anatomical level in one FTS-treated mouse in which only a very small lesion was discernible (B).

FIG. 7 is a graph showing the time course of neurological recovery of mice after CHI. Mice were subjected to CHI and their neurological severity scores (NSSs) were assessed 1 hour after the injury. Immediately thereafter they were treated with 5 mg/kg FTS or vehicle (n=10 per group). The NSS was re-evaluated between 24 hrs and 7 days after CHI. ΔNSS is shown for both groups. **P<0.001 compared to the control group, at all time points tested.

FIG. 8 is a collection of phase contrast and fluorescent images, illustrating that FTS protects hippocampal neuronal cells in culture against glutamate toxicity. Primary hippocampal neuronal cultures were exposed to 25 μM FTS 24 h prior to the addition of glutamate. Controls received the vehicle (0.1% DMSO). The cells were then exposed to 200 μM glutamate for 30 min. The medium was replaced by glutamate-free medium and 24 h later the cell were subjected to the Live/Dead assay. Typical phase contrast images (A) and fluorescent (live cells) images (B) in the same fields are shown for control (no glutamate), glutamate and glutamate plus FTS treated cultures.

FIG. 9 is a collection of phase contrast and fluorescent images, illustrating that FTS protects cortical neuronal cells in culture against glutamate toxicity. Primary cortical neuronal cultures were exposed to 25 μM FTS 24 h prior to the addition of glutamate. Controls received the vehicle (0.1% DMSO). The cells were then exposed to 200 μM glutamate for 30 min. The medium was replaced by glutamate-free medium and 24 h later the cell were subjected to the Live/Dead assay. Typical phase contrast images (A) and fluorescent (live cells) images (B) in the same fields are shown for control (no glutamate), glutamate and glutamate plus FTS treated cultures.

FIG. 10 is a bar graph illustrating that FTS protects hippcampal and cortical neuronal cells in culture against glutamate toxicity. Primary hippocampal and cortical neuronal cultures were exposed to 25 μM FTS 24 h prior to the addition of glutamate. Controls received the vehicle (0.1% DMSO). The cells were then exposed to 200 μM glutamate for 30 min. The medium was replaced by glutamate-free medium and 24 h later, the cells were subjected to the Live/Dead assay and the number of live cells was then estimated. Results are presented as percent cell death where the number of dead cells in the glutamate treated cultures (glutamate toxicity) was referred to as 100% cell death. * p=0.015, ** p=0.028 (student test).

### DETAILED DESCRIPTION

By the term “neurodegenerative disorder”, it is meant any disorder in which progressive loss of neurons occurs either in the peripheral nervous system or in the central nervous system. Examples of neurodegenerative disorders include: chronic neurodegenerative diseases such as Alzheimer's disease, Parkinson's disease, Huntington's chorea, diabetic peripheral neuropathy, amyotrophic lateral sclerosis (Lou Gehrig's disease); aging; and acute neurodegenerative disorders including: stroke, traumatic brain injury, schizophrenia, peripheral nerve damage, hypoglycemia, spinal cord injury, epilepsy, anoxia and hypoxia.

Some embodiments of the present invention are directed to the treatment of traumatic brain or head injuries. In general, traumatic brain injuries occur when the head experiences a sudden physical assault severe enough to cause damage to the brain. Traumatic brain injuries can be either closed or penetrating. A closed head injury (CHI) is one in which the continuity of the scalp and mucous membrane is maintained, thus no breakage in the skull occurs. A penetrating injury, on the other hand, typically involves skull breakage. Sudden and violent blows to the head may be caused by incidents related to transportation, bicycle riding, scooters, sports and recreation, shaken baby syndrome, falling and violence.

Neurodegenerative disorders such as traumatic brain injuries may be diagnosed by a healthcare practitioner (e.g., medical or veterinary) in accordance with standard medical procedures. For example, symptoms that may aid in a diagnosis of traumatic head or brain injury include poor balance, disorientation, dissociation of thought, rages, black out, garbled speech, memory loss, headache, depression, spinal fluid coming out of the ears and nose, loss of consciousness, dilated or unequal pupils, loss of eye movement, respiratory failure, semi-comatose state, coma, impaired muscle tone and muscle movement, slow pulse, one sided paralysis, slow respiratory rate with an increase in blood pressure, vomiting, lethargy, confusion, inefficient thinking/impaired cognition, inappropriate emotional response, changes in personality, irritability, seizures, nausea and dizziness.

The Ras protein is the on/off switch between hormone/growth factor receptors and the regulatory cascading that result in cell division. For Ras to be activated (i.e., turned on) to stimulate the regulatory cascades, it must first be attached to the inside of the cell membrane. Ras antagonist drug development aimed at blocking the action of Ras on the regulatory cascades has focused on interrupting the association of Ras with the cell membrane, blocking activation of Ras or inhibiting activated Ras. Galectin-1 and galectin-3, β-galactoside-binding proteins (Brewer, et al., Biochim. Biophys. Acta 1572:255-62 (2002); Gabius, et al., Eur. J. Biochem. 243:543-76 (1997); Camby, et al., Brain Pathol. 11:12-26 (2001); and Liu, et al., Biochim. Biophys. Acta 1572:263-73 (2002)), have been shown to interact with Ras-GTP (Elad-Sfadia, et al., J. Biol. Chem. 277 (40):37169-75 (2002); Elad-Sfadia, et al., J. Biol. Chem. (in press 2004); and Paz, et al., Oncogene 20:7486-93 (2000)). Galectin-1 strengthens membrane association of H-Ras-GTP. H-Ras-GTP and K-Ras-GTP recruit galectin-1 from the cytosol to the cell membrane resulting stabilization of Ras-GTP (Paz, et al., supra., Elad-Sfadia, et al., (2002), supra.), clustering of H-Ras-GTP and galectin-1 in nonraft microdomains (Prior, et al., J. Cell Biol. 160:165-170 (2003)), enhancement of the Ras signal to extracellular signal-regulated kinase (ERK), and increased cell transformation (Paz, et al., supra., Elad-Sfadia, et al., (2002), supra.). K-Ras-GTP recruits galectin-3 from the cytosol to the cell membrane and enhances Ras transformation (Elad-Sfadia, et al., (2004), supra.). Computational analysis identified a farnesyl-binding pocket in galectin-1 (Rotblat, et al., Cancer Res. 64:3112-18 (2004)). Replacement of a critical amino acid in this pocket yeilded a dominant interfering mutant that, unlike galectin-1 (which co-operates with Ras), extricates oncogenic H-Ras from the membrane and inhibits Ras transforming activity. These observations indicate the significance of Ras/galectin-1 and of Ras/galectin-3 interactions in Ras biology. The farnesyl-binding pocket in galectin-1 is thus a target for the Ras inhibitor FTS that displaces Ras binding to galectin-1 (Elad-Sfadia, et al., (2002) and (2004) supra.; Rotblat, et al., (2004), supra.).

The details of the approaches to development of Ras antagonists are reviewed in Kloog, et al., Exp. Opin. Invest. Drugs 8(12):2121-2140 (1999). Thus, by the term “ras antagonist”, it is meant any compound or agent that prevents its proper localization in the cell membrane, targets the active form of ras by dislodging it from the cell membrane, or prevents activated Ras from signaling to downstream Ras effectors.

Some Ras antagonists useful in connection with the present invention are represented by formula I: 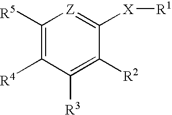
wherein
R¹ represents farnesyl, geranyl or geranyl-geranyl;
Z represents C—R⁶ or N;
   R² represents H, CN, the groups COOR⁷, SO₃R⁷, CONR⁷R⁸, COOM, SO₃M and SO₂NR⁷R⁸, wherein R⁷ and R⁸ are each independently hydrogen, alkyl or alkenyl, and wherein M is a cation (e.g., Na⁺ or K⁺);
   R³, R⁴, R⁵ and R⁶ are each independently hydrogen, carboxyl, alkyl, alkenyl, aminoalkyl, nitroalkyl, nitro, halo, amino, mono- or di-alkylamino, mercapto, mercaptoalkyl, axido, or thiocyanato;
      X represents O, S, SO, SO₂, NH or Se; and
      the quaternary ammonium salts (e.g., methyl and ethyl) and N-oxides of the compounds of formula (I) wherein Z is N.

Other Ras antagonists useful in connection with the present invention are represented by formula II: 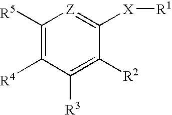
wherein
R¹ represents farnesyl, geranyl or geranyl-geranyl;
Z represents C—R⁶;
   R² represents H, CN, the groups COOR⁷, SO₃R⁷, CONR⁷R⁸, COOM, SO₃M and SO₂NR⁷R⁸, wherein R⁷ and R⁸ are each independently hydrogen, alkyl or alkenyl, and wherein M is a cation;
   R³, R⁴, R⁵ and R⁶ are each independently hydrogen, carboxyl, alkyl, alkenyl, aminoalkyl, nitroalkyl, nitro, halo, amino, mono- or di-alkylamino, mercapto, mercaptoalkyl, axido, or thiocyanato; and
X represents O, S, SO, SO₂, NH or Se.

Yet other Ras antagonists useful in connection with the present invention are represented by formula III: 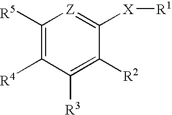
wherein
R¹ represents farnesyl, geranyl or geranyl-geranyl;
Z represents C—R⁶;
   R² represents CN, the groups COOR⁷, SO₃R⁷, CONR⁷R⁸, COOM, SO₃M and SO₂NR⁷R⁸, wherein R⁷ and R⁸ are each independently hydrogen, alkyl or alkenyl, and wherein M is a cation;
   R³, R⁴, R⁵ and R⁶ are each independently hydrogen, carboxyl, alkyl, alkenyl, aminoalkyl, nitroalkyl, nitro, halo, amino, mono- or di-alkylamino, mercapto, mercaptoalkyl, axido, or thiocyanato; and
      X represents O, S, SO, SO₂, NH or Se.

These compounds represent farnesyl-thiosalicylic acid (FTS) (e.g., S-trans, trans-FTS) and its analogs. In embodiments wherein R² represents H, R³ is preferably a carboxyl group.

The structures of FTS and two preferred analogs are as follows:
(i) FTS: 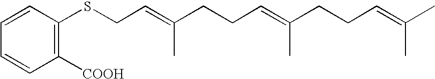
(ii) 2-chloro-5-farnesylaminobenzoic acid (NFCB): 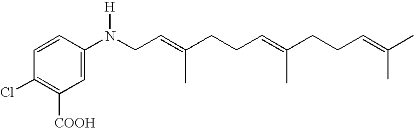
(iii) farnesyl thionicotinic acid (FTN):

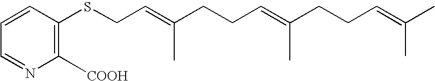

Yet other FTS analogs embraced by formula I include 5-fluoro-FTS, 5-chloro-FTS, 4-chloro-FTS and S-farnesyl-thiosalicylic acid methyl ester (FMTS). Structures of these compounds are set forth below.

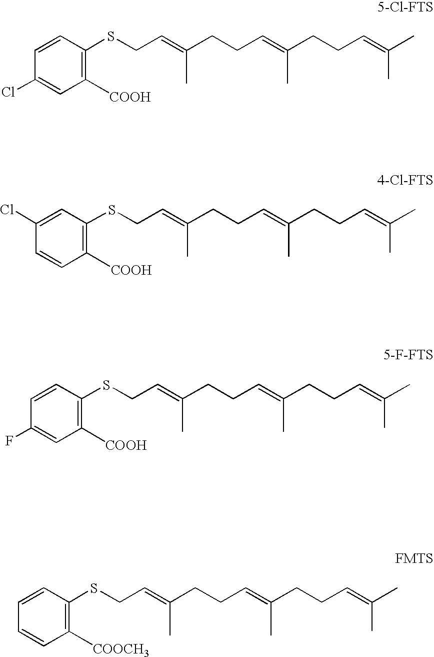

Other ras antagonists that may be useful in the present invention are disclosed in Marciano, et al., 1995, J. Med. Chem. 38, 1267; Haklai, et al., 1998, Biochemistry 37, 1306; Casey, et al., Proc. Natl. Acad. Sci. USA 86, 8323; Hancock, et al., 1989, Cell 57, 1167 and Aharonson, et al., 1998, Biochim. Biophys. Acta. 1406, 40.

A particularly preferred agent is FTS. The mechanism of FTS action is known. In earlier studies, applicant demonstrated that FTS inhibits Ras-dependent cell growth in vitro and inhibits both receptor-mediated and constitutively active Ras-mediated ERK activation (Kloog et al., 1999). FTS affects Ras-membrane interactions, dislodging Ras from its anchorage domains and facilitating its degradation (Haklai et al., 1998). It thus seems that Ras must be anchored to the inner leaflet of the cell membrane in order to receive and transmit signals (Shields et al., 2000), and that FTS, acting directly on saturable Ras-anchorage sites in the cell membrane, prevents Ras from associating with these sites (Niv et al., 2002). Ras, when in its GTP-bound active state, interacts with sites distinct from those with which inactive GDP-bound Ras interacts (Niv et al., 2002; Prior et al., 2001), and that FTS affects primarily the interactions of Ras-GTP with the cell membrane (Haklai et al., 1998; Kloog et al., 1999). This shows that FTS acts as an activity-dependent drug and may explain why FTS was shown not to be toxic and have no adverse side effects in animals (Kloog et al., 1999). FTS and related Ras inhibitors destabilize the proper attachment of Ras to the cell membrane, which is promoted by the Ras carboxy terminal S-farnesyl cysteine required for Ras signaling. FTS has the ability to disrupt the interactions of Ras with the cell membrane in living cells without cytotoxicity. Without intending to be bound by any particular theory of operation, it is believed that the mechanism of action involves a dual effect on membrane Ras where initially (within 30 min) FTS releases Ras from constraints on its lateral mobility which is followed by release of Ras into the cytoplasm and then by Ras degradation. Other Ras antagonists useful in the present invention may be identified by using the cell free membrane assays and cellular assays described in WO 98/38509, WO 02/29031, which teaches assays for identifying antagonists of Ras/galectin-1, and the mouse model of head injury disclosed in Chen, et al. (1996).

In general, the Ras antagonists are substantially insoluble in water and saline solutions such as PBS. Thus, salified agents [e.g., an NA⁺, K⁺ or NH⁺ form] formulated with an organic solvent such an alkyl gallates and butylated hydroxyanisole containing lecithin and/or citric acid or phosphoric acid, are suitable for parenteral administration, which is a preferred mode of administering the ras antagonists, such as in the case of acute head trauma and other embodiments where the patient is physically or mentally incapable of taking the Ras antagonist orally. Administration may be transdermal as well.

In other embodiments, however, oral administration is acceptable and even preferred. Ras antagonists such as FTS and its analogs may be formulated in cyclodextrin. This technology is the subject of U.S. Pat. Nos. 5,681,828 and 5,935,941. Cyclodextrins are a group of compounds consisting of, or derived from, the three parent cyclodextrins—alpha-, beta- and gamma-cyclodextrins. Alpha-, beta- and gamma-cyclodextrins are simple oligosaccharides consisting of six, seven or eight anhydroglucose residues, respectively, connected to macrocyles by alpha (1 to 4) glycosidic bonds. Each of the glucose residues of a cyclodextrin contains one primary (O6) and two secondary hydroxyls (O2 and O3), which can be substituted, for example, methylated. Cyclodextrins solubilize insoluble compounds into polar media by forming what is known as an inclusion complex between the cyclodextrin and the insoluble compound; cyclodextrin solubilization power is directly proportional to the stability of the complex. Inclusion complexes are non-covalent associations of molecules in which a molecule of one compound, called the host, has a cavity in which a molecule of another compound, called a guest is included. Derivatives of cyclodextrins are used as the hosts, and the insoluble compound is the guest.

Briefly, the Ras antagonist is salified and dissolved in an appropriate solvent, and then added to a solution of methylated cyclodextrin in PBS. The result of the solution is sterilized and then the solvent is removed. To prepare a formulation suitable for oral administration, the resultant cyclodextrin/FTS complex is mixed with a suitable binder and then pressed into buccal tablets. These tablets dissolve when held in the mouth against the mucus membrane. It is believed that as the tablet dissolves, the cyclodextrin particles touch the membrane and the drug is released and is passed across the membrane of the mouth into the bloodstream. Alternatively, the cyclodextrin/Ras antagonist complex can be reconstituted into an appropriate solution or emulsion suitable for parenteral (e.g., intramuscular, intravenous or subcutaneous) administration.

In other embodiments, the Ras antagonists may also be formulated in compressed tablets, in capsules, and in hard or soft gelcaps, containing pharmaceutically acceptable binders, lubricants, disintegrants, gelling agents, and solubilizing liquids e.g., starch, lactose, microcrystalline cellulose, hydroxypropylcellulose, polyvinylpyrrolidone, magnesium stearate, talc, stearic acid, low molecular weight polyethylene glycols, vegetable oils and other excipients and carrier materials known to those skilled in the art of pharmaceutical formulations.

The term “treatment” is broadly intended to mean the retardance or inhibition or even reversal of the progression or course of a neurological disorder, or amelioration of at least one symptom associated therewith. Without intending to be bound by any particular theory of operation, it is believed that the present invention works on a cellular level by inhibiting or protecting nerve cells from deterioration and cell death arising from a neurodegenerative disorder (termed “neuroprotection” or “reduction of a neurological deficit”), and on a biochemical level by reducing levels of Ras-GTP or reducing loss of NMDAR binding associated with a neurodegenerative disorder or in the case of some neurodegenerative disorders, by reducing glutamate-mediated toxicity. In general, amounts of the Ras antagonist effective for treatment are from about 1.5 mg/kg to about 40 mg/kg of patient weight, and preferably from about 2 mg/kg to about 20 mg/kg. In general, the Ras antagonists may be administered as a single dose (e.g., injection), or once, twice or three times a day, once every two days or three times per week for extended therapy.

The frequency of the administrations, and the duration of same, will vary. These parameters may be determined by a health care provider in accordance with established clinical procedures, taking into consideration factors such as, but not limited to: age, severity of injury, and the age, weight and overall physical condition of the patient.

The present invention will now be described by way of the following examples. They are presented solely for purposes of illustration, and are not intended to limit the invention in any way. For instance, the examples provide protocols for determining whether a given ras antagonist provides “treatment” of a neurological disorder.

### EXAMPLE 1

### Demonstration of Neuroprotective Effect of FTS on CHI Mice

Drug Treatments

FTS was a gift from Thyreos (Newark, N.J., U.S.A.). Its purity, assessed by thin-layer chromatography, [¹H]-NMR, and mass spectral analysis, was >95%. FTS powder was diluted in chloroform (35.8 mg/mL FTS=0.1 M) and kept in aliquots. Aliquots were evaporated under nitrogen and their contents (per aliquot) were dissolved in 4 μL of absolute ethanol and 7 μL of NaOH, to which 890 μL of phosphate-buffered saline (PBS) was then added. Each mouse received 0.1 mL of this solution at a dosage of 5 mg/kg. This dose was selected for the present study based on our earlier experiments in other models in which dose response for inhibition of Ras was studied. This dose was found to be both, effective and safe. MK-801 was purchased from Sigma, dissolved in saline, and injected at a dose of 1 mg/kg. The drugs were injected intraperitoneal (i.p.) 1 hour after CHI.

Pharmacokinetics of FTS in the Brain

Farnesyl 1-³H-thiosalicylic acid ([³H]-FTS), 12.5 Ci/mmol, 1 mCi/mL, was purchased from American Radiolabeled Chemicals (ARC; St. Louis, Mo., U.S.A.). The labeled drug was isotopically diluted with unlabeled FTS. Mice (ICR strain) were injected i.p. with 0.1 ml of this FTS solution (14.8 μCi, 351 nmol, 3 mg/kg). At each time point (2.5, 5, 10, 20, 60, and 120 min) after the injection, two mice were killed and their brains were removed. The forebrains were washed in PBS, weighed and homogenized, and samples were counted in a scintillation fluid using an LKB β-counter with automatic correction for quenching. Data are expressed as [³H]-FTS in dpm/g tissue as a function of time.

Animals and Trauma

The study was approved by the Institutional Animal Care Committee of Hadassah Medical Center and the Hebrew University. Sixty male C57bl mice weighing 25 to 35 g were used. CHI was induced under ether anesthesia, as previously described (Chen et al., 1996) and modified (Yatsiv et al., 2002). Briefly, after induction of ether anesthesia the skull was exposed by a midline longitudinal incision. A tipped Teflon cone was placed in the mid-coronal plane above the left anterior frontal area, 1 mm lateral to the midline. A weight (74 g) was dropped onto the cone (from a height of 15 cm), resulting in a focal injury. After trauma, animals received supporting oxygenation with 100% O₂ for no longer than 2 min and were then returned to their cages. Sham-injured mice were anesthetized and their skin was incised, but they were not subjected to CHI.

Ras-GTP and Phospho-ERK Assays

In the first experiment mice were sacrificed 10, 30, or 120 min after sham-injury or CHI to assess levels of Ras=GTP and phospho=ERK. In the second set of experiments, the animals received FTS (5 mg/kg i.p.) or vehicle, 1 hr after CHI and sham-injured mice received the vehicle (n=4 per group). The mice were sacrificed 2 or 24 hrs after CHI, their brains were removed, and cortical tissue samples adjacent or contra-lateral to the site of injury were homogenized in homogenization buffer containing protease inhibitors and 0.5% Triton X100 as described (Haklai et al., 1998). Debris was removed by centrifugation and protein in the extract was determined with the aid of the Bio-Rad protein assay (Bio-Rad Laboratories, GmbH). Total Ras protein was determined by Western immunoblotting of 30 μg of protein with 1:2500 pan-Ras Ab (Oncogene Research Products, followed by 1:7500 peroxidase goat anti-mouse IgG (Haklai et al., 1998). For determination of GTP-bound Ras in protein samples (1 mg), Ras-GTP was pulled down by glutathione-S-transferase fused to the Ras-binding domain of Raf (GST-RBD) which binds Ras-GTP only. The GST-RBD-Ras-GTP was pulled down with glutathione-agarose beads, and Ras was then determined by immunoblotting with pan-Ras Ab as described above, followed by enhanced chemiluminescence (ECL; Amersham Pharmacia Biotech, Piscataway, N.J., U.S.A) (Paz et al., 2000). The bands were quantified by densitometry with Image Master VDS-CL (Amersham Biotech) using TINA 2.0 software (Ray Tests).

ERK and phospho-ERK were determined in 30 μg of brain extract proteins by immunoblotting, ECL, and densitometry (Paz et al., 2000). ERK immunoblots were incubated with 1:2000 rabbit anti-ERK1/2 Ab (Santa-Cruz) and then with 1:1000 peroxidase-goat anti rabbit IgG. Phospho-ERK immunoblots were incubated with 1:10,000 mouse anti-phospho-ERK Ab (Sigma) and then with 1:10,000 peroxidase-goat anti-mouse IgG.

Neurobehavioral Evaluation

The neurological severity score (NSS) is a tool for assessing an animal's functional status. It is based on the ability of the animal to perform different motor and behavioral tasks representing motor ability, balancing, and alertness (Beni-Adani et al., 2001). Scores range from zero, achieved by healthy uninjured animals, to a maximum of 10, indicating severe neurological dysfunction, with failure of all tasks. The NSS obtained 1 hr after trauma reflects the initial severity of injury and is inversely correlated with neurological outcome. Animals were evaluated 1 hour after CHI, and again at 24 and 48 hrs and at 5 and 7 days. To assess the effect of FTS on neurological recovery, mice were randomly assigned to treatment with either FTS or vehicle (n=10 per group), administered immediately after the initial NSS evaluation. Each animal was assessed by an observer who was blinded to the treatment it had received. The extent of recovery (ΔNSS) was calculated as the difference between the NSS at 1 hr and at any subsequent time point. Thus, a higher ΔNSS reflects better recovery. This parameter therefore serves as a tool for evaluation of drug effects.

Quantitative Autoradiography and Assessment of Infarct Volume

Seven days after CHI, vehicle-treated (n=5) and FTS-treated (n=4) mice were decapitated and their brains were removed (within 1 minute), frozen in powdered dry ice, and kept at −70° C. until used. Consecutive 20-μ cryostat sections of the whole forebrain were cut in the coronal plane, and one in every ten sections (i.e. at intervals of 200μ) was thaw-mounted onto coated microscope glass slides.

NMDAR autoradiography was performed as described (Bowery et al., 1988), with some modifications. After being pre-washed for 30 minutes in 50 mM Tris-acetate buffer at pH 7.4, the sections were incubated for 2.5 hrs at room temperature in the same buffer containing 10 nM [³H]MK-801, 30 μM glutamate, and 10 μM glycine (200 μL per section). Nonspecific binding was determined in the presence of excess (100 μM) unlabeled MK-801. At the end of incubation, the sections were dipped for 5 seconds in ice-cold buffer, washed for 90 minutes in cold fresh buffer, and then dipped in ice-cold distilled water. The dried tissue sections were exposed to tritium-sensitive film accompanied by commercial calibrated tritium standard scales (Amersham). After exposure for 36 days, the films were developed in Kodak D-19, fixed, and dried. The sections were then stained with cresyl violet for anatomical region placement according to a mouse brain atlas (Paxinos and Franklin, 1997) and for identification of lesions.

Quantitative Image Analysis

The films were scanned and digitized using PhotoShop and a large bed Umax scanner, and saved in tiff format for accessibility to NIH image software. Using NIH Image routines, the standard curve was measured and used to calibrate regional brain measurements. Morphometry routines were used to measure the lesion area on each section where it was visible. The volume was calculated by multiplying the summed lesion areas by the distance between sections (0.2 mm).

Statistical Analysis

Values of NSS are expressed as means±SD, and analyzed using the Kruskall-Wallis nonparametric test. NMDAR binding densities in various brain regions ipsi- and contra-lateral to the trauma were compared by a side X region ANOVA followed by regional post hoc analyses. Ras, ERK, and phospho-ERK were quantified as described above, expressed as means±SD, and analyzed using Student's t-test. A P value of <0.05 is considered significant.

Mice subjected to CHI were treated systemically 1 hour later with FTS (5 mg/kg) or vehicle. After 1 hour, Ras-GTP in the contused hemisphere showed a significant (3.8-fold) increase, which was strongly inhibited by FTS (82% inhibition) or by the NMDA-receptor antagonist MK-801 (53%). Both drugs also decreased active (phosphorylated) extracellular signal-regulated kinase. FTS prevented the CHI-induced reduction in NMDAR binding in cortical, striatal, and hippocampal regions, measured by [³H]-MK-801 autoradiography, and decreased lesion size by 50%. It also reduced CHI-induced neurological deficits, indicated by the highly significant (P<0.0001) 60% increase in extent of recovery. Thus, FTS provided long-term neuroprotection after CHI, rescuing NMDAR binding in the contused hemisphere and profoundly reducing neurological deficits. These findings suggest that non-toxic Ras inhibitors such as FTS may qualify as neuroprotective drugs.

Results

Systemically Injected FTS Accumulates in the Brain

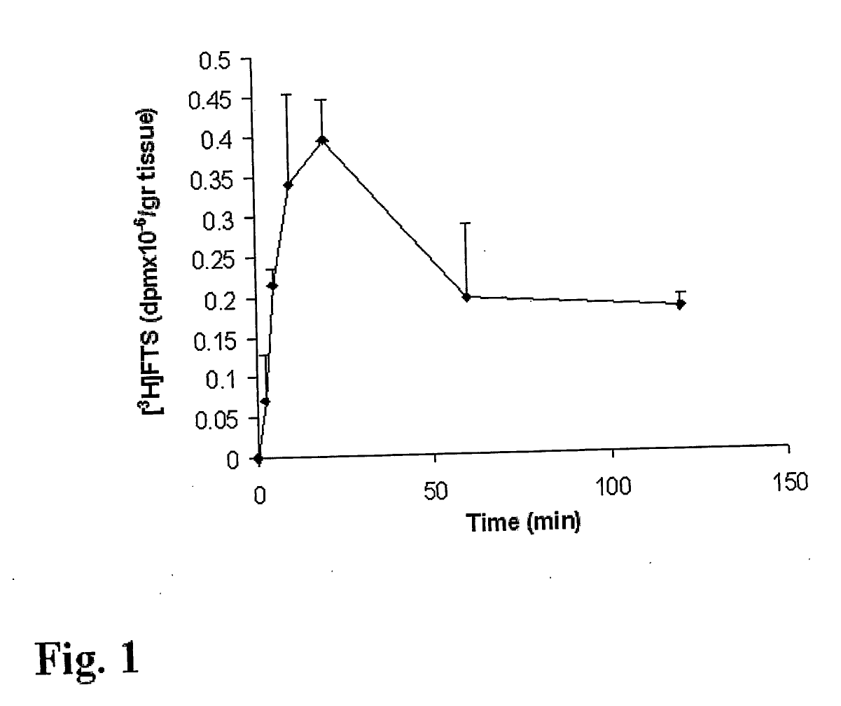

First it was necessary to determine whether FTS can enter the brain, and if so, whether it can exist there in the micromolar concentration range previously found to be relevant to its efficacy as a Ras inhibitor in vitro (Kloog et al. 1999). To this end, we performed pharmacokinetic experiments in which mice received [³H]-FTS (3 mg/kg, i.p.), and the amounts of labeled inhibitor in the brain were then determined by counting radioactivity in samples of brain homogenates. Results of a typical experiment demonstrate that FTS enters the mouse brain, reaching peak amounts within 20 to 30 minutes and remaining relatively high for at least 2 hrs (FIG. 1). Based on the specific activity of [³H]-FTS and assuming a homogeneous distribution in the brain, we estimated that the peak concentration of FTS was 4.5 μM. Thus, under the conditions employed here, it appears that a pharmacologically relevant concentration of FTS in the brain was achieved.

The CHI-Induced Increase in Brain Ras-GTP is Inhibited by FTS

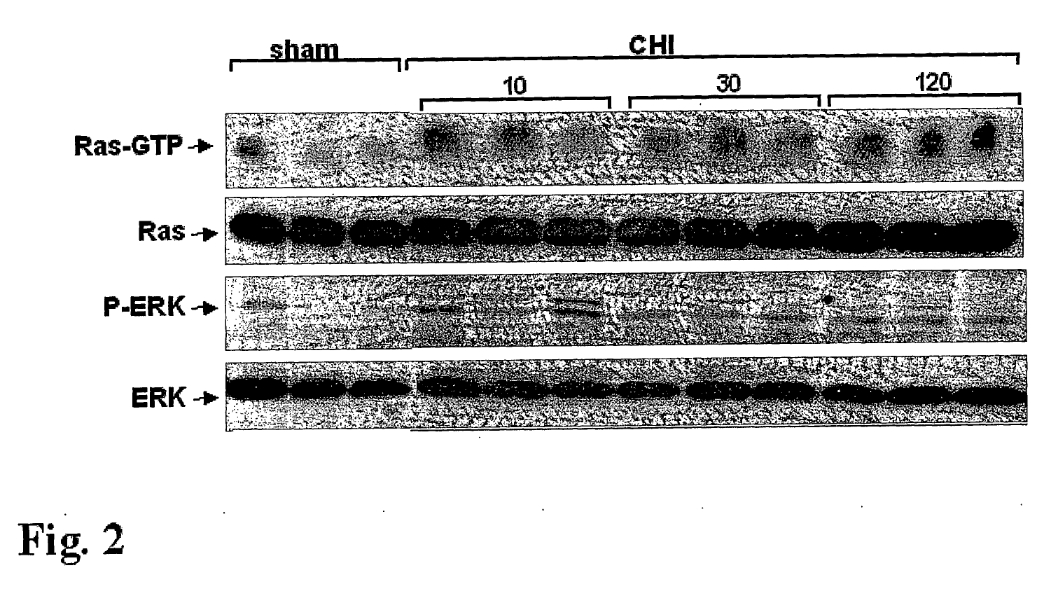

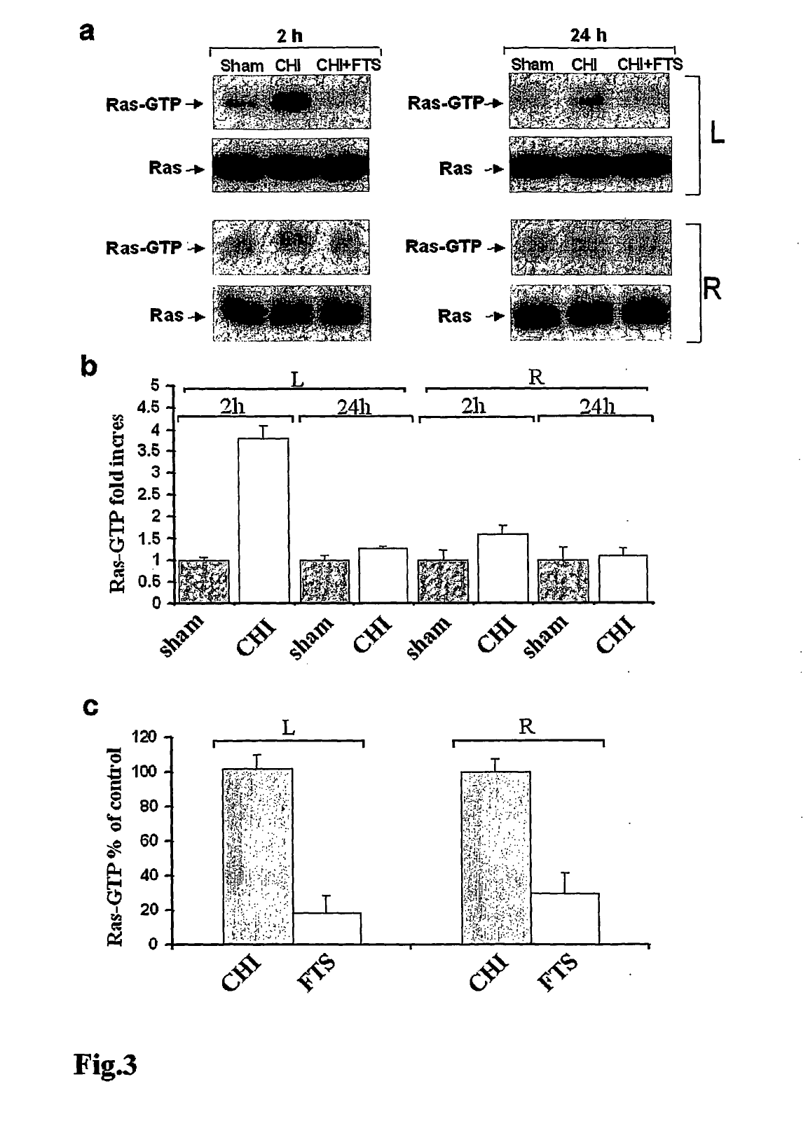

Next we examined whether CHI induces activation of the Ras pathway, as it does in other trauma models (Ferrer et al., 2002). Since previous studies indicated changes in basal levels of active phospho-ERK after traumatic brain injury, we first determined the temporal changes in the levels of active Ras-GTP and of phospho-ERK after CHI. Mice were sacrificed at 10 min, 30 min and 2 h after CHI or sham surgery (n=3/time point) and the left (contused) hemisphere was analyzed by Western blot. It can be seen that under these conditions the basal levels of Ras-GTP and of phospho-ERK are relatively low in the sham animals (FIG. 2). CHI induced an increase in Ras-GTP and phospho-ERK, observed already at 10 min after injury and remaining relatively high even 2 hrs later (FIG. 2). Here too, only small variations in the levels of Ras-GTP and phospho-ERK were observed between the triplicates (FIG. 2). Notably, there were no changes in the amounts of total Ras or total ERK proteins. In a second set of experiments, the effect of CHI on the total amounts of Ras and of active Ras-GTP in the brains of injured mice was examined, 2 and 24 hrs after the injury, in the contused (left) and contra-lateral hemispheres. FIG. 3a shows that although CHI did not alter the total amount of Ras in either hemisphere, there was a marked increase in Ras-GTP 2 hrs after CHI compared to the amounts of Ras-GTP observed in the sham-injured mice. The increase was significantly more pronounced in the contused (left) hemisphere (3.8-fold increase, P=0.016) than in the contra-lateral hemisphere (1.6-fold increase) (FIG. 3b). In both hemispheres, however, the increase in Ras-GTP was observed 2 but not 24 hrs after the injury, indicating the transient nature of this response.

We next examined in a second set of experiments whether the Ras inhibitor FTS can alter the CHI-induced increase in Ras-GTP levels in the brain. One hour after CHI, mice were injected i.p., either with vehicle or with 5 mg/kg FTS. This dose of FTS was found in previous studies to suppress neoplasticity in a number of animal models without inadvertent side effects (Jansen et al., 1999; Kloog et al., 1999). The amounts of Ras and Ras-GTP were determined 2 and 24 hrs after injury. The results of a typical experiment show that the CHI-induced increase in Ras-GTP observed 2 hrs after the injury was strongly inhibited by FTS (FIG. 3a, left panels). This was seen in both the contused and the contra-lateral hemispheres. As shown in FIG. 3c, the extents of inhibition recorded in the left and right hemispheres were 82±10% and 70±12%, respectively (n=3). Notably, FTS had almost no effect on Ras-GTP levels when assessed 24 hrs after CHI (FIGS. 3a and 3b), when the amounts of active Ras were already as low as in the sham-injured mice. Also, FTS had no effect on the total amount of Ras in the brain (FIG. 3). Taken together, these results support earlier studies on other brain-insult models (Ferrer et al., 2002; Mandell et al., 2001; Mori et al., 2002; Otani et al., 2002) in which activation of the Ras/MAPK pathway was demonstrated, and are consistent with an FTS action mechanism that acts preferentially on active GTP-bound Ras (Kloog et al., 1999).

MK-801, Like FTS, Reduces the Amounts of Ras-GTP and Phospho-ERK in the Brains of CHI Mice

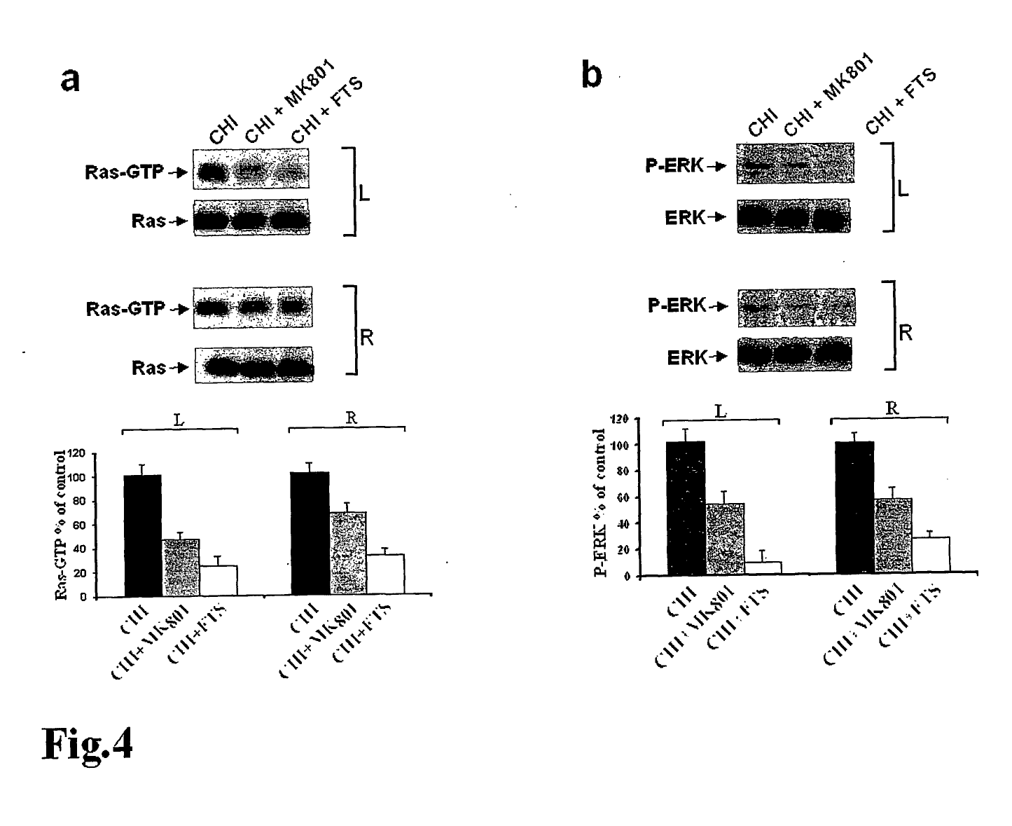

To determine whether the CHI-induced increase in Ras-GTP was associated with NMDAR functions, we assayed Ras-GTP in CHI mice injected i.p. with FTS (5 mg/kg) or the NMDAR antagonist MK-801 (1 mg/kg) 1 hr after injury. Amounts of Ras-GTP were determined 2 hrs after CHI, a time point at which we observed a marked increase in Ras activation (FIG. 4). MK-801 partially inhibited the transient CHI-induced increase in Ras-GTP in both the contused (left) and the contra-lateral hemispheres (FIG. 4a). The calculated extent of inhibition (FIG. 4a) was 53±8% in the left hemisphere and 31±5% in the right (n=4). These results show that the increase in Ras-GTP in the brains of the CHI mice was associated, at least in part, with activation of NMDAR.

In the set of experiments described above, we also determined the effects of MK-801 and FTS on the amounts of activated (phosphorylated) ERK in the brains of the injured mice. Active ERK and total ERK protein were assayed 2 hrs after CHI. Neither inhibitor altered the amounts of total ERK (FIG. 4b). However, treatment with each of the inhibitors resulted in a decrease in phospho-ERK in both the contused (left) and the contra-lateral hemispheres (FIG. 4b). FTS treatment reduced phospho-ERK by 90±9% in the left hemisphere and by 73±8% in the right hemispheres (n=4), and treatment with MK-801 reduced it by 46±8% and 43±5%, respectively (n=4). These results show that the inhibitory effects of both the NMDAR antagonist MK-801 and the Ras inhibitor FTS on Ras-GTP were also manifested in the Ras-dependent Raf/MEK/ERK cascade.

Effect of FTS on NMDAR Binding

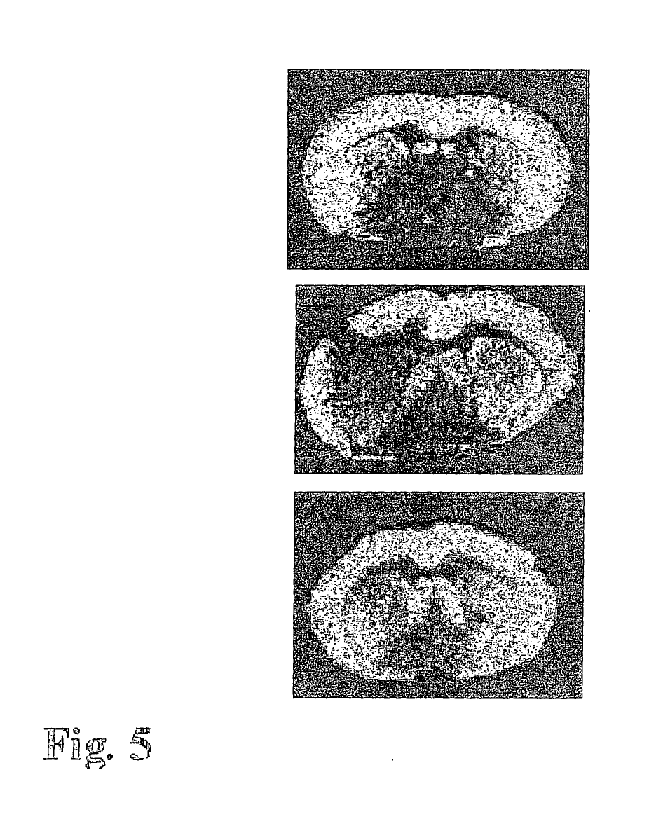

The results described above showed that in CHI, as in other types of trauma, the Ras/MAPK pathway is activated, the activation is at least partially dependent on NMDAR, and the Ras inhibitor FTS strongly inhibits the CHI-induced activation of the Ras/MAPK pathway in the brain. In light of these results and the reported participation of the Ras/MAPK cascade in excitotoxicity (Ferrer et al., 2002), we examined whether treatment with FTS exerts neuroprotective effects. Binding of [³H]-MK-801 to NMDAR was taken to be a measure of NMDA glutamate receptive neurons. As expected, there were no differences in regional MK-801 binding between the left and right side of the brain in sham treated animals (Table 1A). In agreement with previous reports (Biegon et al., 2002; Sihver et al., 2001), it was found that the binding of [³H]-MK-801 to NMDA glutamate receptors in the brain was substantially decreased after CHI, although the extents of decrease were not uniform: ANOVA with repeated measures revealed a significant effect of side (P<0.0001) and a significant side X region interaction (P<0.001) (Table 1B). NMDAR levels in the contralateral (right) hemisphere followed the known regional distribution pattern (Bowery et al., 1988) also seen in the sham-injured animals, with the largest amounts in the hippocampus and cortex. The contralateral hemisphere showed a trend towards lower binding in the frontal motor cortex, but this was not statistically significant. The largest reductions (>20%, or significant at P<0.05 by post-hoc analysis, or both) were observed in regions close to the lesion, including the perilesion area (>40% decrease), parietal cortex, perirhinal cortex, piriform cortex, frontal motor cortex, and dorsal striatum (Table 1B, FIG. 5). More moderate reductions, which were not statistically significant on post-hoc analysis (13-20%), were seen in the ventral striatum and hippocampal CA3 and CA1 fields. Treatment with FTS completely reversed the effect of trauma on the binding of [³H]-MK-801 to the NMDA receptors, as indicated by the finding that ANOVA with repeated measures showed no significant difference between sides and no significant interaction (P=0.5, Table 1B, FIG. 5). Complete reversal of the trauma effect was seen in the piriform cortex, perirhinal cortex, posterior cingulate cortex amygdala, frontal motor cortex, and all dorsal hippocampal fields. A non-significant trend towards lower binding in the ipsilateral hemisphere of the FTS-treated animals was seen only in the parietal cortex and striatum (Table 1B).

Importantly, the animals whose brains were processed for NMDA autoradiography and histology underwent neurological evaluation (see below) and the two groups did not differ in initial injury severity as assessed by the neurological severity score 1 hr after the injury. The median NSS was 6 and the range was 6-7 in both the vehicle and FTS treated groups, and median recovery after seven days (expressed as ΔNSS) was 0 in the vehicle treated mice as compared to 2 in the FTS treated mice (p<0.05, Mann-Whitney test).

Effect of FTS on Lesion Volume

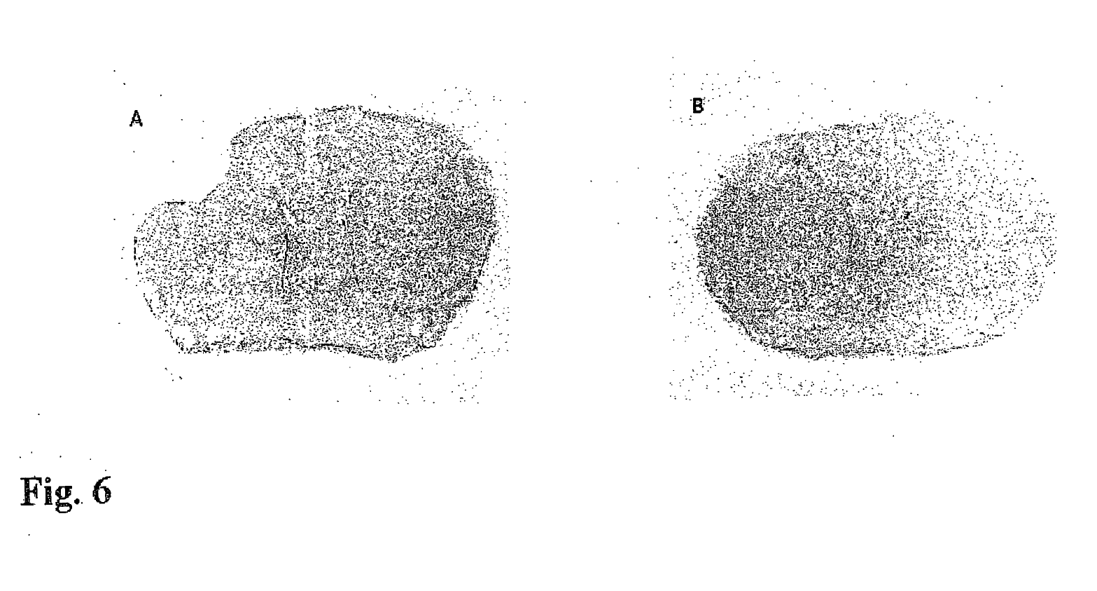

We next examined the effect of FTS on the size of the CHI-induced lesion. This model results in progressive tissue loss and cavitation of the cortex in the injured side that reaches a stable size within 3-7 days. A distinct lesion was indeed observed in all of the mice that were subjected to CHI in this study. As expected, the lesion was located in the left fronto-parietal cortex. The mean lesion volume calculated in the control mice was 155±41 μL (mean±SD of 5 animals, range 113 to 211 μL). FTS treatment resulted in a trend towards a reduction in lesion volume by almost 50%, to 87±53 μL (mean±SD of 4 animals, range 37 to 161, p<0.07, Student's t-test, two tailed). The range of lesions is illustrated in FIG. 6, with the largest cross sectional representation, from a vehicle treated animal shown in FIG. 6A and the section with the smallest lesion, from an FTS treated animal, shown in FIG. 6B.

Effect of FTS on Functional Recovery

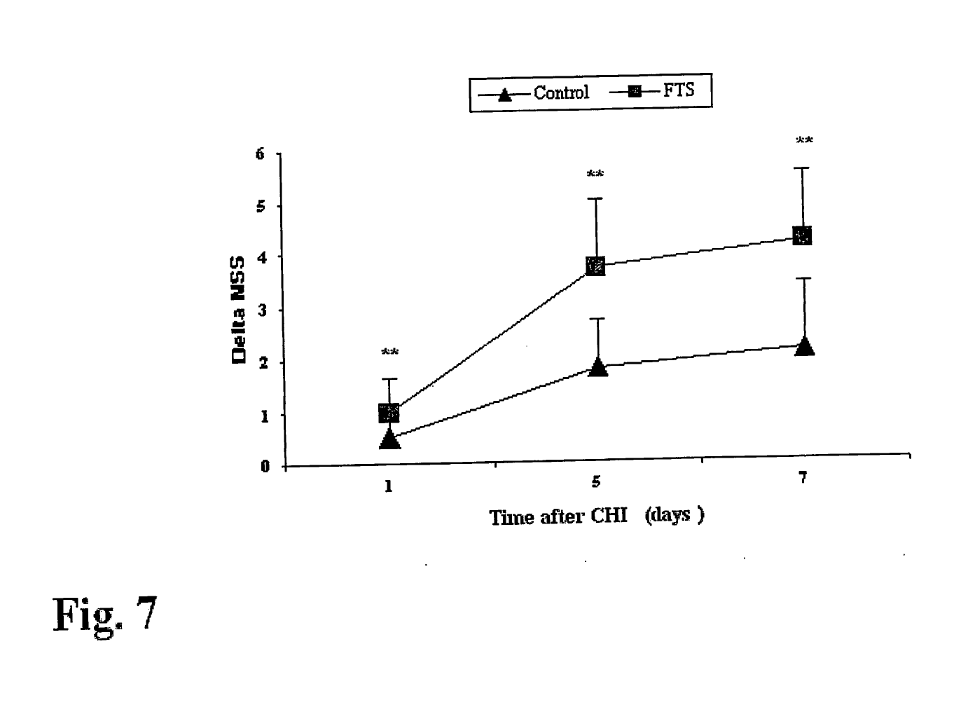

The results described above indicated that FTS exerted a profound neuroprotective effect after CHI in mice. It was therefore of interest to examine whether these FTS-induced effects were also manifested in a decrease in the neurological impairment induced by CHI. The extent of neurological damage was determined, as described (Beni-Adani et al., 2001) (also see Materials and Methods), in terms of the neurological severity score (NSS), which was first evaluated 1 hour after the injury. The mice were then divided into vehicle-treatment (control) and FTS-treatment groups (n=10 per group), ensuring that the severity of injury in the two groups was similar (mean NSS±SD=6.9±0.38 and 6.7±0.3, respectively). Immediately thereafter the mice received either FTS (5 mg/kg, i.p.) or vehicle, and the NSS was then evaluated at different time points and both the spontaneous and the drug-related recovery (in terms of ΔNSS) in the two groups were compared. As shown in FIG. 7, a significantly better recovery was observed as early as 24 hrs after injury in the FTS-treated mice. This effect was maintained for up to 7 days and became even more pronounced over time (P<0.0001 Mann Whitney). The mean ΔNSS values recorded on day 7 after injury were 4.2 in the FTS-treated mice and 1.7 in the controls (FIG. 7). Thus, single-dose treatment with FTS provided a robust, long-lasting beneficial effect that reduced the CHI-induced neurological deficits by 60% (P<0.0001).

Our results show that after CHI in mice, the Ras inhibitor FTS exerts robust neuroprotective effects. This was evident from the better neurological recovery observed in the FTS-treated mice than in vehicle-treated controls, with a highly significant, long-lasting improvement of 60% of neurological status seen even at 7 days after trauma, as well as from the observed rescue of the binding of [³H]-MK-801 to NMDAR and the smaller size of lesions recorded in the brains of FTS-treated injured mice. This demonstrates that inhibition of active Ras by a non-toxic Ras inhibitor can confer neuroprotection and lead to a better neurological recovery after traumatic brain injury. The present results also support the possible development of FTS as a brain-active Ras inhibitor. They imply that FTS crosses the blood-brain barrier, that a pharmacologically relevant concentration of the inhibitor (4.5 μM) is rapidly achieved (FIG. 1), and that FTS inhibits the CHI-induced transient increase in active Ras-GTP and in active phospho-ERK in the brain (FIGS. 3 and 4).

The well-documented release of glutamate after CNS injury is a critical event, which is followed by activation of NMDAR and accumulation of intracellular calcium (Faden et al., 1989). Calcium influx through the NMDAR activates the Ras/ERK pathway (Chen et al., 1998), and ERK in the brain is activated in response to various NMDAR-related stimuli, including long-term potentiation (English and Sweatt, 1996), long-term memory (Brambilla et al., 1997), visual stimulation (Kaminska et al., 1999), associative learning (Atkins et al., 1998) and ischemia (Farnsworth et al., 1995). Knockout mice for the brain-specific Ras exchange factor Ras-GEF, which activates Ras through binding to calcium/calmodulin (Farnsworth 1995), indeed exhibit impaired long-term potentiation and memory consolidation (Brambilla et al., 1997). It is still not clear, however, how NMDAR activation causes an increase in Ras-GTP.

The early increase in Ras-GTP observed after CHI (FIG. 2) correlates well with the increase in Ras-GTP after an excitotoxic insult (Ferrer et al., 2002) and with the time course of the increase in glutamate release under similar conditions (Faden et al., 1989). The greater increase in Ras-GTP in the injured hemisphere (3.8 fold) than in the contra-lateral hemisphere (1.6 fold) would indicate that the release of glutamate occurs mainly at the site of injury.

While not intending to be bound by any particular theory of operation, applicants, in view of the above considerations, believe that the most likely sequence of events after CHI is the release of glutamate followed by NMDAR activation, subsequently resulting in increased calcium influx and Ras activation. It is worth noting that although both inhibitors, MK-801 and FTS, inhibited the CHI-induced increase in Ras-GTP and in phospho-ERK, they acted through entirely different mechanisms. MK-801 (which blocks NMDAR and calcium influx) would either prevent a receptor-mediated exchange of GDP for GTP on Ras or decrease a receptor-mediated inhibition of Ras-GAP activity. FTS, however, is known to act mainly on membrane Ras once it has become active (GTP-bound) through the action of Ras exchange factors (reviewed in: Kloog et al., 1999). Thus, the decrease in Ras-GTP observed with MK-801 treatment represents inhibition of exchange, or decrease in GTP hydrolysis by Ras, or both, whereas the decrease observed with FTS treatment represents a direct effect of the inhibitor on membrane association of the active Ras-GTP formed as a consequence of CHI and activation of NMDAR. The preferential effect of FTS on Ras-GTP without affecting the total amount of Ras (FIGS. 3 and 4) is consistent with the above-mentioned mechanism of drug action.

It is important to note that in addition to regulating the Raf/MEK/ERK pathway, Ras directly and indirectly regulates many other signaling cascades, including phosphoinositide 3-kinase pathways, the Ral-GTPase pathways, the Rac and Rho GTPases, and the p38 and Jun kinase pathways (Shields et al., 2000).

Whether or not the CHI-induced increase in Ras-GTP results in activation of a multitude of Ras effectors, our results show that both the increase in Ras-GTP (FIGS. 1 and 2) and the loss of NMDAR binding (FIG. 6 and Table 2) were inhibited by treatment with FTS. The post-CHI loss of NMDAR, which is believed to contribute to neurological deficits (Biegon et al., 2002; Friedman et al., 2001; Miller et al., 1990; Sihver et al., 2001), was strongly inhibited by FTS and was indeed manifested in a decrease in the CHI-induced neurological deficits (FIG. 7). A significant, long-lasting improvement in neurological status was recorded between 24 hrs and 7 days in the FTS-treated mice. During the entire period of follow-up the ΔNSS was greater in the FTS-treated mice than in the vehicle-treated mice, and this effect, although already significant 24 hrs after CHI, became more pronounced with time. A similar pattern to that found in mice was also observed in rats (not shown).

Many of the clinical signs of CHI, including memory impairment (Chen et al., 1996), are probably manifestations of functional loss of NMDAR and NMDA-receptive neurons. NMDAR are indeed vulnerable to traumatic, ischemic, and inflammatory brain damage, and this effect is reversed by early administration of MK-801 (Biegon et al., 2002; Friedman et al., 2001; Miller et al., 1990; Sihver et al., 2001). FTS is capable of complete reversal of NMDAR loss in the traumatized hemisphere. Taken together with the findings that both FTS and MK-801 treatments reduced the relatively large amounts of Ras-GTP observed in the brains of CHI mice, as well as the improvement in neurological status after FTS treatment, these observations strongly suggest a direct protective effect of FTS on NMDA-receptive neurons. FTS also significantly reduces the mean lesion area.

The significantly stronger effect of FTS than of MK-801 on Ras-GTP (inhibition of 70-82% compared to 31-53%) suggests that some of the positive effects of the Ras inhibitor might be mediated by inhibition of NMDAR-independent processes. One such process might be neuroinflammation, which is among the early post-traumatic responses sustained over a long period (7 days and more) (Feuerstein et al., 1998). Active Ras participates in neuroinflammatory responses, and mechanical trauma induces Ras-dependent astroglial MAPK activation (Dalakas, 1995). Indeed, many forms of brain injuries, including trauma and inflammation, induce astrogliosis and activation of astroglia (Mandell et al., 2001).

In conclusion, the neuroprotective effect of FTS, expressed in the results of behavioral testing and in the rescue of NMDA-receptive neurons, supports the role of Ras-GTP activation as an early upstream signal in the late consequence of traumatic brain injury, and suggests that early inhibition of this pathway or intracellular events further downstream could provide new strategies for the management of head injury.

TABLE 1
NMDA-receptor density in various brain regions
A: In sham - treated mice

| Region | Right | Left |
|---|---|---|
| Parietal cortex | 6.99 ± 1.08 | 7.61 ± 1.0 |
| Perirhinal | 6.58 ± 0.72 | 7.01 ± 0.67 |
| cortex | | |
| Frontal motor | 9.62 ± 2.2 | 10.6 ± 2.4 |
| cortex | | |
| Piriform cortex | 7.02 ± 0.46 | 7.88 ± 0.8 |
| Dorsal striatum | 6.03 ± 0.9 | 6.11 ± 0.6 |
| Ventral striatum | 5.93 ± 0.8 | 5.95 ± 0.74 |
| Hippocampus CA3 | 6.98 ± 1.1 | 7.28 ± 1.02 |
| Hippocampus CA1 | 9.44 ± 2.45 | 10.1 ± 2.35 |

B: Seven days after CHI without or with FTS treatment

| | CHI-Control | | CHI + FTS | |
|---|---|---|---|---|
| Region | Right | Left | Right | Left |
| Parietal | 7.28 ± 1.6 | 4.93 ± 1.21* | 7.04 ± 1.46 | 5.69 ± 1.18 |
| cortex | | | | |
| Perirhinal | 6.72 ± 0.94 | 4.67 ± 0.62* | 5.97 ± 0.88 | 5.92 ± 0.72 |
| cortex | | | | |
| Frontal motor | 7.78 ± 2.89 | 5.83 ± 2.44* | 6.63 ± 1.7 | 6.32 ± 2.12 |
| cortex | | | | |
| Piriform | 5.59 ± 1.83 | 4.4 ± 1.01* | 5.57 ± 0.84 | 5.65 ± 1.54 |
| cortex | | | | |
| Dorsal | 6.11 ± 3.29 | 4.79 ± 1.95 | 5.45 ± 1.72 | 4.32 ± 0.32 |
| striatum | | | | |
| Ventral | 5.99 ± 2.19 | 5.1 ± 2.15 | 5.21 ± 0.54 | 4.06 ± 1.82 |
| striatum | | | | |
| Hippocampus | 8.18 ± 3.92 | 6.73 ± 1.48 | 7.23 ± 0.42 | 7.69 ± 0.96 |
| CA3 | | | | |
| Hippocampus | 9.01 ± 3.85 | 8.17 ± 2.28 | 9.25 ± 0.32 | 9.85 ± 1.34 |
| CA1 | | | | |

Results are mean ± SD of right (contra-lateral) and left (ipsilateral) hemisphere readings from A: 4 sham-injured mice and B: 5 CHI-vehicle treated and 4 CHI-FTS treated mice.
Data are expressed as nCi of [³H]-MK-801 specifically bound/mg.
*P < 0.05 compared to the contra-lateral (uninjured) hemisphere.

### REFERENCES

1. Atkins C M, Selcher J C, Petraitis J J, Trzaskos J M, and Sweatt J D (1998) The MAPK cascade is required for mammalian associative learning Nat Neurosci 1:602-609.
2. Beni-Adani L, Gozes I, Cohen Y, Assaf Y, Steingart R A, Brenneman D E, Eizenberg O, Trembolver V, and Shohami E (2001) A peptide derived from activity-dependent neuroprotective protein (ADNP) ameliorates injury response in closed head injury in mice J Pharmacol Exp Ther 296:57-63.
3. Biegon A, Alvarado M, Budinger T F, Grossman R, Hensley K, West M S, Kotake Y, Ono M, and Floyd R A (2002) Region-selective effects of neuroinflammation and antioxidant treatment on peripheral benzodiazepine receptors and NMDA receptors in the rat brain J-Neurochem 82:924-934.
4. Bowery N G, Wong E H, and Hudson A L (1988) Quantitative autoradiography of [3H]-MK-801 binding sites in mammalian brain Br J Pharmacol 93:944-954.
5. Brambilla R, Gnesutta N, Minichiello L, White G, Roylance AJ, Herron C E, Ramsey M, Wolfer D P, Cestari V, Rossi-Arnaud C, et al. (1997) A role for the Ras signalling pathway in synaptic transmission and long-term memory Nature 390:281-286.
6. Chen H J, Rojas-Soto M, Oguni A, and Kennedy M B (1998) A synaptic Ras-GTPase activating protein (p135 SynGAP) inhibited by CaM kinase II Neuron 20:895-904.
7. Chen Y, Constantini S, Trembovler V, Weinstock M, and Shohami E (1996) An experimental model of closed head injury in mice: pathophysiology, histopathology, and cognitive deficits J Neurotrauma 13:557-568.
8. Dalakas M C (1995) Basic aspects of neuroimmunology as they relate to immunotherapeutic targets: present and future prospects Ann Neurol 37:S2-13.
9. Elad-Sfadia G, Haklai R, Ballan E, Gabius H J, and Kloog Y (2002) Galectin-1 augments Ras activation and diverts Ras signals to Raf-1 at the expense of phosphoinositide 3-kinase J-Biol-Chem 277:37169-37175
10. English J D, and Sweatt J D (1996) Activation of p42 mitogen-activated protein kinase in hippocampal long term potentiation J-Biol-Chem 271:24329-24332.
11. Faden A I, Demediuk P, Panter S S, and Vink R (1989) The role of excitatory amino acids and NMDA receptors in traumatic brain injury Science 244:798-800.
12. Farnsworth C L, Freshney N W, Rosen L B, Ghosh A, Greenberg M E, and Feig L A (1995) Calcium activation of Ras mediated by neuronal exchange factor Ras-GRF Nature 376:524-527.
13. Ferrer I, Blanco R, Carmona M, Puig B, Dominguez I, and Vinals F (2002) Active, phosphorylation-dependent MAP kinases, MAPK/ERK, SAPK/JNK and p38, and specific transcription factor substrates are differentially expressed following systemic administration of kainic acid to the adult rat Acta Neuropathol (Berl) 103:391-407.
14. Feuerstein G Z, Wang X, and Barone F C (1998) The role of cytokines in the neuropathology of stroke and neurotrauma Neuroimmunomodulation 5:143-159.
15. Friedman L K, Ginsberg M D, Belayev L, Busto R, Alonso O F, Lin B, and Globus M Y (2001) Intraischemic but not postischemic hypothermia prevents non-selective hippocampal downregulation of AMPA and NMDA receptor gene expression after global ischemia Brain Res Mol Brain Res 86:34-47.
16. Fukunaga K, and Miyamoto E (1998) Role of MAP kinase in neurons Mol Neurobiol 16:79-95.
17. Haklai R, Gana-Weisz G, Elad G, Paz A, Marciano D, Egozi Y, Ben Baruch G, and Kloog Y (1998) Dislodgment and accelerated degradation of Ras Biochemistry 37:1306-1314
18. Jansen B, Heere-Ress E, Schlagbauer-Wadl H. Halaschek-Wiener J, Waltering S, Moll I, Pehamberger H, Marciano D, Kloog Y, and Wolff K (1999) Farnesylthiosalicylic acid inhibits the growth of human Merkel cell carcinoma in SCID mice J Mol Med 77:792-797
19. Kaminska B, Kaczmarek L, Zangenehpour S, and Chaudhuri A (1999) Rapid phosphorylation of Elk-1 transcription factor and activation of MAP kinase signal transduction pathways in response to visual stimulation Mol Cell Neurosci 13:405-414.
20. Kloog Y, Cox A D, and Sinensky M (1999) Concepts in Ras-directed therapy Exp. Opin. Invest. Drugs 8:2121-2140
21. Kochanek P, Clark R, Ruppel R, Adelson P, Bell M, Whalen M, Robertson C, Satchell M, Seidberg N, Marion D, and Jenkins M (2000) Biochemical, cellular and molecular mechanisms in the evolution of secondary damage after severe traumatic brain injury in infants and children: lessons learned from the bedside Pediatr Crit Care 1:4-19
22. Laurer H L, and McIntosh T K (1999) Experimental models of brain trauma Curr Opin Neurol 12:715-721.
23. Mandell J W, Gocan N C, and Vandenberg S R (2001) Mechanical trauma induces rapid astroglial activation of ERK/MAP kinase: Evidence for a paracrine signal Glia 34:283-295.
24. Miller L P, Lyeth B G, Jenkins L W, Oleniak L, Panchision D, Hamm R J, Phillips L L, Dixon C E, Clifton G L, and Hayes R L (1990) Excitatory amino acid receptor subtype binding following traumatic brain injury Brain-Res 526:103-107.
25. Morganti-Kossmann M C, Rancan M, Stahel P F, Kossmann T. (2000) Inflammatory response in acute traumatic brain injury: a double-edged sword. Curr Opin Crit Care 8:101-105.
26. Mori T, Wang X, Jung J C, Sumii T, Singhal A B, Fini M E, Dixon C E, Alessandrini A, and Lo E H (2002) Mitogen-activated protein kinase inhibition in traumatic brain injury: in vitro and in vivo effects J Cereb Blood Flow Metab 22:444-452.
27. Niv H, Gutman O, Kloog Y, and Henis Y I (2002) Activated K-Ras and H-Ras display different interactions with saturable nonraft sites at the surface of live cells J-Cell-Biol 157:865-872
28. Otani N, Nawashiro H, Fukui S, Nomura N, Yano A, Miyazawa T, and Shima K (2002) Differential activation of mitogen-activated protein kinase pathways after traumatic brain injury in the rat hippocampus J Cereb Blood Flow Metab 22:327-334.
29. Paxinos G, and Franklin K B J (1997) The mouse brain in sterotaxic coordinates, 2nd edn, Academic Press)
30. Paz A, Haklai R, Elad G, Ballan E, and Kloog Y (2000) Galectin-1 binds H-Ras to mediate Ras membrane anchorage and cell transformation Oncogene 20:7486-7493
31. Prior I A, Harding A, Yan J, Sluimer J, Parton R J, and Hancock J F (2001) GTP-dependent segregation of H-ras from lipid rafts is required for biological activity Nature Cell Biology 3:368-375
32. Rosenblum K, Berman D E, Hazvi S, Lamprecht R, and Dudai Y (1997) NMDA receptor and the tyrosine phosphorylation of its 2B subunit in taste learning in the rat insular cortex J-Neurosci 17:5129-5135.
33. Scheffzek K, Ahmadian M R, Kabsch W, Wiesmuller L, Lautwein A, Schmitz F, and Wittinghofer A (1997) The Ras-RasGAP complex: structural basis for GTPase activation and its loss in oncogenic Ras mutants Science 277:333-337
34. Shapira Y, Yadid G, Cotev S, Niska A, and Shohami E (1990) Protective effect of MK801 in experimental brain injury J Neurotrauma 7:131-139.
35. Shields J M, Pruitt K, McFall A, Shaub A, and Der C J (2000) Understanding Ras: ‘it ain't over 'til it's over’ Trends-Cell-Biol 10:147-154
36. Shohami E, Ginis I, and Hallenbeck J M (1999) Dual role of tumor necrosis factor alpha in brain injury Cytokine Growth Factor Rev 10:119-130.
37. Sihver S, Marklund N, Hillered L, Langstrom B, Watanabe Y, and Bergstrom M (2001) Changes in mACh, NMDA and GABA(A) receptor binding after lateral fluid-percussion injury: in vitro autoradiography of rat brain frozen sections J-Neurochem 78:417-423.
38. Sosin D M, Sniezek J E, and Waxweiler R J (1995) Trends in death associated with traumatic brain injury, 1979 through 1992. Success and failure Jama 273:1778-1780.
39. Yatsiv I, Morganti-Kossmann M C, Perez D, Dinarello C A, Novick D, Rubinstein M, Otto V I, Rancan M, Kossmann T, Redaelli C A, et al. (2002) Elevated intracranial IL-18 in humans and mice after traumatic brain injury and evidence of neuroprotective effects of IL-18-binding protein after experimental closed head injury J Cereb Blood Flow Metab 22:971-978.

### EXAMPLE 2

### FTS Protects Nerve Cells from Glutamate Toxicity

Preparation of Primary Neuronal Cultures from Embryonic Rat Brain

Hippocampal and cortical neuronal cultures were prepared from embryonic rat brain essentially as described by Mattson (Mattson P M, Barger S W, Begley J M and Mark R J, Methods Cell Biol 1995; 46: 187-216). Briefly, Sprague Dawley embryos (17-18 days of gestation) were removed and their brains were dissected under the hood and kept in cold in sterile HEPES buffered Hank's balanced salt solution (HBBS) lacking Ca²⁺ and Mg²⁺, containing 10 μg/ml gentamicin sulfate. The brain regions under study were dissected and cells were dissociated by mild 0.25% trypsin, counted and the dissociation buffer was replaced by culture medium as detailed in Mattson. The cells were plated on poly-L-lysine (10 g/ml solution) coated 24 well plates in Neurobasal medium (Gibco, Grand Island, N.Y. # 2110 3-049) prepared as detailed in Mattson. Hippocampal or cortical cells were plated at a density of 5×10⁵ cells per well in 24 well plates and grown in 1 ml medium. Cultures were a kept in humidified 95%, 5% air/CO₂ incubator at 37 C for 7 days and then used for the experiments.

Experimental Protocol

Cells were treated with 25 μM FTS either 24 h prior to glutamate (200 μM) treatment or immediately after glutamate treatment. Assays were performed in triplicates. Twenty-four hours after the glutamate treatment, the cells were subjected to viability-cytotoxicity assay using the Live/Dead reagent kit (L-7013, Molecular Probes) essentially as described by the manufacturer. Fluorescent images for live cells (Syto 10, green) and of dead cells (DEAD red) were collected with appropriate filters within 1 h of staining. The number of live cells was estimated by counting the green-labeled cells in 3 fields in each well. The number of live cells in each of the triplicate experimental samples was averaged. These values were used to estimate the percent of cell death comparing the glutamate treated cells (with or without FTS) to untreated controls. The read labeled dead cells could not be used for accurate estimation of cell death under the conditions used because unsynchronized cell-death leads to disintegration of dead cells resulting in inaccurate estimation of the number of dead cells.

Results

In the typical experiments, either hippocampal or cortical neuronal culture was exposed to 25 μM FTS 24 h prior to the addition of glutamate. Controls received the vehicle (0.1% DMSO) which itself had no toxic effects. The cells were then exposed to 200 μM glutamate for 30 min. The medium was replaced by glutamate-free medium and 24 h later the cells were subjected to the Live/Dead assay. Under the conditions used, it was found that glutamate induced 25-30% death of both the hippocampal and the cortical, primary neurons. This level of cell death was used as a reference point in all experiments.

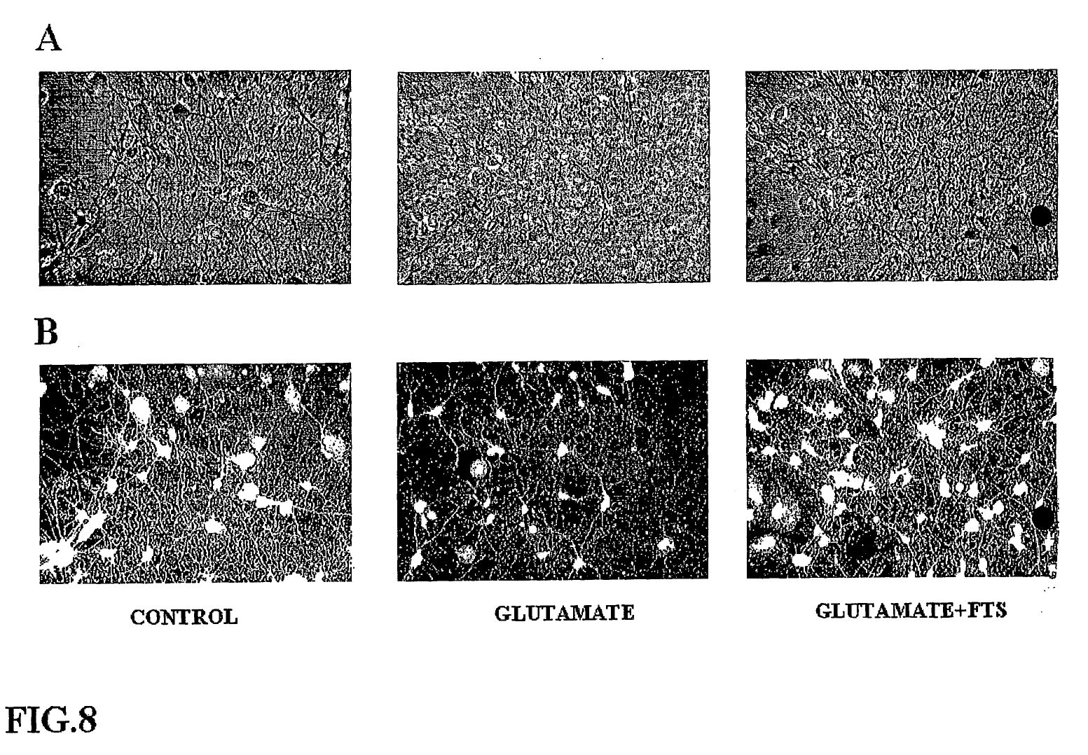

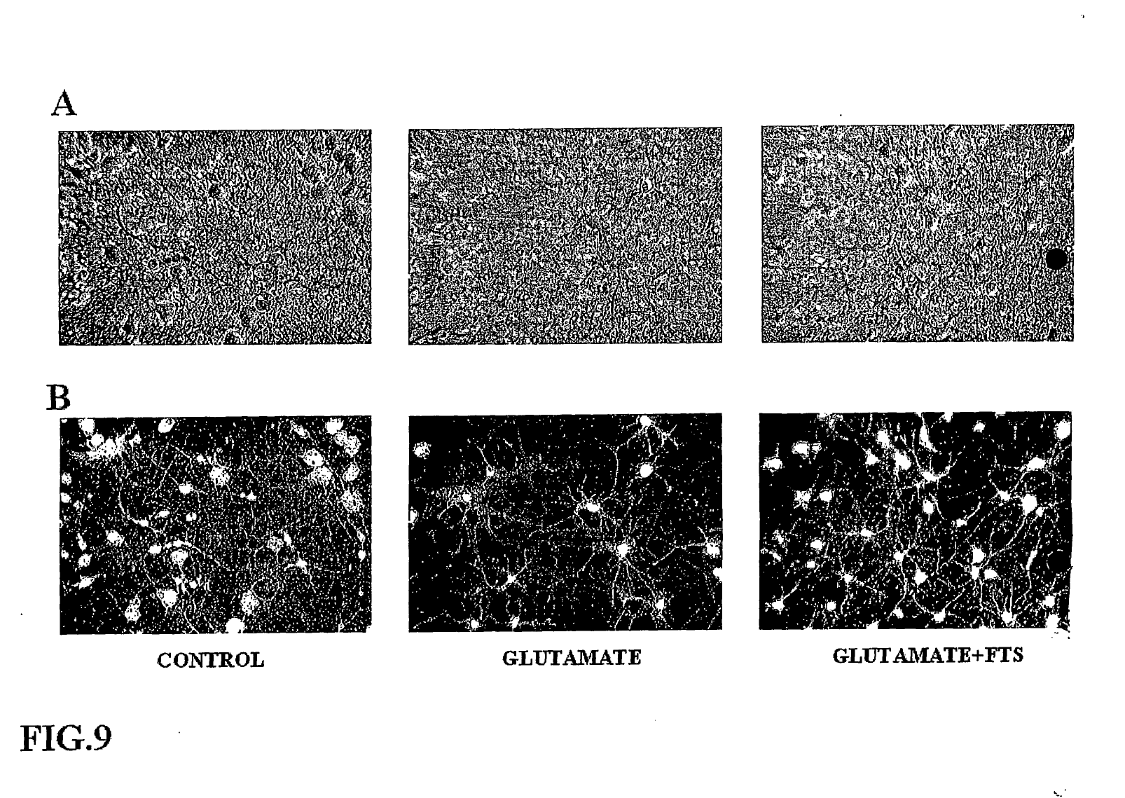

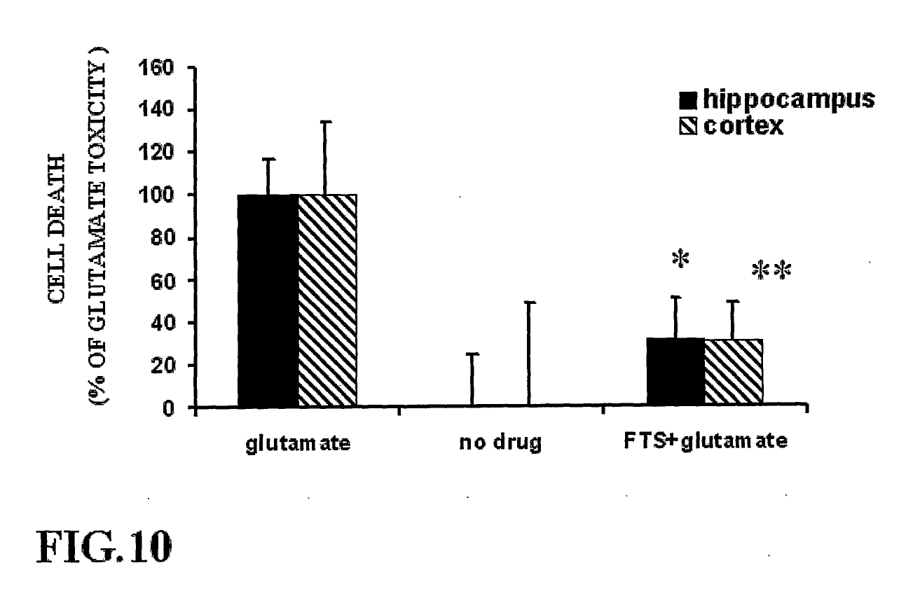

Typical phase contrast images and green fluorescent images of control, glutamate treated and glutamate plus FTS hippocampal cultures are shown in FIG. 8. As shown, glutamate treatment induced a significant decrease in the number of live cells where the clear disintegration of neuritis is observed. In the presence of FTS the toxic effect of glutamate was markedly reduced (FIG. 8). Typical phase contrast images and green fluorescent images of control, glutamate treated and glutamate plus FTS cortical cultures are shown in FIG. 9. Here too, the glutamate treatment induced a significant decrease in the number of live cells where the clear disintegration of neuritis is observed. FTS also reduced the toxic effect of glutamate in the cortical cultures (FIG. 9). In separate experiments, it was found that FTS alone had no toxic effects on the cultured hippocampal or cortical neurons. The protective effects of FTS against the glutamate neurotoxicity were estimated by direct counting of the live (green labeled) cells. As shown in FIG. 10, in the presence of FTS only 30% of the cells died as compared to the 100% cell death of the glutamate treated cells. This indicates that FTS exhibited a strong (70%) neuroprotective effect against the glutamate toxicity. Similar results were obtained when FTS was added immediately after exposure to glutamate indicating that FTS did not act on the NMDA receptors.

These results demonstrate the utility of FTS as a therapeutic drug to protect neuronal cell loss in stroke, ischemia, anoxia, hypoxia, Wernicke-Kosakoff's related dementia (alcohol induced dementia), hematoma and epilepsy and other related diseases.

### EXAMPLE 3

### Neuroprotection—FTS, Rat Model

Sabra (strain of the Hebrew University) rats (200-220 gr) were used. A weight (200 g) was dropped on the cone fixed on the exposed skull at the site (frontal left cortex) designated for injury (from a height of 20 cm), resulting in a focal injury. A maximal Neurological Severity Score (NSS) of 17 indicates severe neurological dysfunction, with failure of all tasks, whereas a score of zero is achieved by healthy uninjured animals. The NSS at 1 h after trauma reflects the initial severity of injury and is inversely correlated with neurological outcome. Animals were evaluated 1 hour after CHI, and later, at 24 hrs, 48 hrs, 5 days and 7 days. To assess the effect of FTS on neurological recovery, rats were randomly assigned to either vehicle or FTS treatment, given immediately after the initial NSS evaluation (at 1 hr after CHI). These assessments were performed by an observer blinded to the kind of treatment the animals have received. The extent of recovery (ΔNSS) was calculated as the difference between NSS at 1 hr and that at any later time point:
ΔNSS (at time t)=NSS (1 h)−NSS (t).

Thus, the greater ΔNSS reflects greater recovery, and this parameter serves as a tool for evaluation of drug effects.

As shown in table 2, a significantly better recovery was observed as early as 24 hrs after injury in the FTS-treated mice. This effect was maintained for up to 7 days and became even more pronounced over time (P=0.019 Mann Whitney). Thus, single-dose treatment with FTS provided a robust, long-lasting beneficial effect that reduced the CHI-induced neurological deficits in the rat model.

TABLE 2
Effect of FTS on functional recovey (ΔNSS) of rats after CHI

| | Neurological | | | |
|---|---|---|---|---|
| | Severity | | | |
| | Score (NSS) | ΔNSS = NSS (1 h) − NSS (time) | | |
| | 1 h | 24 h | 3 d | 7 d |
| CHI- | 9.28 ± 2.75 | 1.28 ± 0.75 | 2.57 ± 0.78 | 3.14 ± 0.90 |
| control | | | | |
| CHI + FTS | 9.71 ± 1.97 | 1.86 ± 1.34 | 3.00 ± 1.29 | 5.14 ± 1.07* |

NSS of rats was evaluated at 1 h, and immediately thereafter, they were treated with vehicle or FTS (5 mg/kg bw, ip). The rats were re-assessed at later time points.
Note that the NSS for rats, although similar in principal to that of mice, is based on 17 tasks (not shown), thus the maximal score is 17.
Values of NSS are represented as mean ± SD of 7 rats.
*p = 0.019 vs control, vehicle-treated rats.

1-24. (canceled)

25. A method of treating a neurodegenerative disorder, comprising administering to a human in need thereof an effective amount of a Ras antagonist.

26. The method of claim 25, wherein the neurodegenerative disorder involves glutamate-mediated toxicity.

27. The method of claim 26, wherein the neurodegenerative disorder is a traumatic head or brain injury, ischemia or stroke.

28. The method of claim 27, wherein the traumatic head or brain injury is a closed head injury.

29. The method of claim 27, wherein the traumatic head or brain injury is a penetrating injury.

30. The method of claim 26, wherein the neurodegenerative disorder is selected from the group consisting of stroke, schizophrenia, peripheral nerve damage, hypoglycemia, spinal cord injury, epilepsy, anoxia and hypoxia.

31. The method of claim 25, wherein the neurodegenerative disorder is a chronic disorder.

32. The method of claim 31, wherein the chronic disorder is selected from the group consisting of Alzheimer's disease, Parkinson's disease, Huntington's chorea, diabetic peripheral neuropathy, amyotrophic lateral sclerosis and aging.

33. The method of claim 25, wherein the Ras antagonist is represented by the formula: 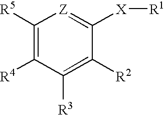
wherein
R¹ represents farnesyl, geranyl or geranyl-geranyl;
Z represents C—R⁶ or N;
R² represents H, CN, the groups COOR⁷, SO₃R⁷, CONR⁷R⁸, COOM, SO₃M and SO₂NR⁷R⁸, wherein R⁷ and R⁸ are each independently hydrogen, alkyl or alkenyl, and wherein M is a cation;
R³, R⁴, R⁵ and R⁶ are each independently hydrogen, carboxyl, alkyl, alkenyl, aminoalkyl, nitroalkyl, nitro, halo, amino, mono- or di-alkylamino, mercapto, mercaptoalkyl, axido, or thiocyanato;
X represents O, S, SO, SO₂, NH or Se; and
the quaternary ammonium salts and N-oxides of the compounds of said formula when Z is N.

34. The method of claim 33, wherein Z represents C—R⁶.

35. The method of claim 34, wherein R² represents CN or a group which is COOR⁷, SO₃R⁷, CONR⁷R⁸, COOM, SO₃M or SO₂NR⁷R⁸.

36. The method of claim 33, wherein the Ras antagonist is farnesyl-thiosalicyclic acid (FTS).

37. The method of claim 33, wherein the Ras antagonist is 2-chloro-5-farnesylaminobenzoic acid (NFCB).

38. The method of claim 33, wherein the Ras antagonist is farnesyl thionicotinic acid (FTN).

39. The method of claim 33, wherein the Ras antagonist is 5-fluoro-FTS.

40. The method of claim 33, wherein the Ras antagonist is 5-chloro-FTS.

41. The method of claim 33, wherein the Ras antagonist is 4-chloro-FTS.

42. The method of claim 33, wherein the Ras antagonist is S-farnesyl-thiosalicylic acid methyl ester.

43. The method of claim 25, wherein the treatment comprises parenteral administration of the Ras antagonist.

44. The method of claim 25, wherein the treatment comprises oral administration of the Ras antagonist.

45. The method of claim 44, wherein the Ras antagonist is administered in a formulation containing a cyclodextrin.

46. The method of claim 25, wherein the effective amount of a Ras antagonist reduces levels of Ras-GTP.

47. The method of claim 25, wherein the effective amount of a Ras antagonist reduces levels of N-methyl-D-aspartate (NMDA) receptive neurons.

48. The method of claim 25, wherein the effective amount of a Ras antagonist reduces glutamate toxicity.
